(19) United States
(12) Patent Application Publication
(10) Pub. No.: US 2002/0170088 A1
(43) Pub. Date: Nov. 14, 2002

(54) Novel auxin binding proteins and uses thereof

(75) Inventor: Thea A. Wilkins, Woodland, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 10/053,200

(22) Filed: Nov. 2, 2001

Related U.S. Application Data
(60) Provisional application No. 60/245,816, filed on Nov. 3, 2000.

(51) Int. Cl. A01H 1/00; C07H 21/04; C12N 5/04

(52) U.S. Cl. 800/278; 435/419; 435/320.1; 536/23.6

(57) ABSTRACT

This invention pertains generally to the production of genetically engineered plants with altered responses to auxin. For example, this invention pertains to a family of novel auxin-binding polypeptides and genetically engineered plants with improved properties due to the expression of these polypeptides.

### STATEMENT AS TO RIGHTS TO INVENTIONS MADE UNDER FEDERALLY SPONSORED RESEARCH AND DEVELOPMENT

Not applicable.

### FIELD OF THE INVENTION

This invention pertains generally to the production of genetically engineered plants with altered phenotypes resulting from modulating auxin responses in the plant. For example, cotton plants with altered fiber properties, including increased cotton fiber length, and greater yield can be produced. In particular, this invention pertains to a family of novel auxin-binding polypeptides (ABPs) and genetically engineered plants expressing recombinant ABPs. These transgenic plants have improved properties due to the expression of the ABP nucleic acids of the invention.

### INTRODUCTION

Auxins are a class of plant growth regulators involved in the control of cell expansion. Although the primary mechanism of auxin action, is not completely understood, it is believed that auxins cause cell wall changes including the simultaneous synthesis and breakdown of cell wall, alter ion flux at the plasma membrane especially potassium and proton flux, permitting turgor-driven cell elongation. Regulation of gene expression by auxin is also thought to be involved in the process of for cell expansion.

The quality of a cotton plant as a textile fiber source is dependent on its fiber length, fineness, and strength. These fibers are actually single-celled outgrowths (called trichomes) from individual epidermal cells in the developing cotton seed. The length and fineness of the fiber largely determines the quality of the resulting cotton thread. The final length of a fiber is the product of the cell's rate of elongation and the total period of elongation. Accordingly, attempts to produce better quality cotton fiber have focused on means to increase cotton fiber cell length, fineness, and strength.

Previous studies to identify the factors that influence fiber length and quality have included attempts to measure the levels of endogenous auxin (indoleacetic acid, IAA) and other growth regulatory hormones, such as abscisic acid or gibberellins, in the plant. Such studies have yielded conflicting results, largely due to problems inherent with the methodology employed for measurement of hormone levels, and have generated academic controversy as to the growth regulatory mechanisms that control cotton fiber elongation. For example, Nayyar (1989) Biochem. Physiol. Pflanzen 185:415-421, studied the relationship between the amounts of endogenous IAA (auxin), abscisic acid and a gibberellin (GA₃) on the rate of fiber elongation in Gossypium arboreum. They found no discernable relationship between IAA and GA₃ levels and the rate of fiber elongation. In vivo levels of auxin remained low during the period of rapid fiber elongation (fiber initiation starts from a day before to a day or two after anthesis, the fiber elongation phase begins immediately thereafter, see, e.g., Basra (1984) Int. Rev. Cytol. 89:65-113). Abscisic acid levels were much higher during the period of rapid fiber elongation when compared with IAA and GA₃. In contrast, other studies have suggested that IAA and GA₃ do indeed play a role in fiber length (see, e.g., Naithani (1982) Physiol. Plant. 54:225-229); Bhardwaj (1985) Indian J. Plant Physiol. 20:140-150).

Because fiber growth and elongation requires rapid cell expansion and synthesis of large amounts of cell wall components (the primary cell wall is laid down during the elongation phase), other investigators have focused on the identification of genes which are expressed during the elongation phase; particularly if they encode polypeptides involved in fiber cell wall growth. For example, Song (1997) Biochimica et Biophysica Acta 1351:305-312, found in Gossypium hirsutum a cotton fiber specific acyl carrier protein cDNA important in rapidly elongating cotton fibers because of its role in membrane lipid synthesis. Other investigations, e.g., John (1992) Proc. Natl. Acad. Sci. USA 89:5769-5773; John (1995) Plant Physiol. 108:669-676; John (1996) Plant Molecular Biol. 30:297-306; Rinehart (1996) Plant Physiol. 112:1331-1341; Orford (1998) Biochimica et Biophysica Acta 1398:342-346, focus on the hope that identification of genes expressed during fiber elongation will eventually lead to an ability to influence cotton cell growth, and hence yield, and fiber quality by recombinant genetic technologies.

The receptor mediating auxin effects in plant cells (e.g. cotton fibers) has not been unequivocally identified. A gene encoding an auxin-binding protein Arabidopsis (ABP1)is a leading candidate for mediating at least some of the mentioned auxin effects (Jones et al. Science 282:1114-11147 (1998)).

To date, no mechanisms responsible for controlling fiber growth and elongation have been definitively identified, and no means to generate a cotton plant with increased fiber length has been found. The present invention fulfills these and other needs.

### SUMMARY OF THE INVENTION

The present invention, for the first time, provides a means to produce plants with altered response to auxin. Auxins are important in controlling cell expansion and effect a variety of phenotypes including overall plant architecture. The invention provides a family of novel cotton auxin-binding polypeptides (ABPs) and genes and nucleic acids which encode these ABPs. In one preferred aspect, the invention provides genetically engineered cotton plants with improved properties due to the in vivo expression of these ABP polypeptides. In particular, the genetically engineered cotton cells and plants of the invention, by virtue of expressing the ABPs of the invention, have enhanced cotton fiber properties. Such properties include, for example, fiber length, yield, fineness, uniformity, color, strength, and the like.

The invention provides an isolated nucleic acid that encodes a polypeptide capable of binding to auxin (indoleacetic acid, IAA), wherein the nucleic acid can hybridize to SEQ ID NO:1 under stringent hybridization conditions. In one embodiment, the stringent hybridization conditions comprise a hybridization step having a salt concentration of about 0.02 molar at pH 7 and a temperature of about 60° C. The nucleic acid can have a sequence as set forth in SEQ ID NO:1.

The invention also provides an expression cassette comprising an ABP nucleic acid sequence operably linked to a promoter, wherein the nucleic acid sequence hybridizes to SEQ ID NO:1 under stringent hybridization conditions. The invention also provides a vector comprising a nucleic acid sequence operably linked to a promoter, wherein the nucleic acid sequence hybridizes to SEQ ID NO:1 under stringent hybridization conditions. In either the expression cassette or the vector, the nucleic acid can have a sequence as set forth in SEQ ID NO:1, or subsequences thereof (particularly subsequences encoding biologically active domains or antigenic fragments of the ABP polypeptide).

The invention further provides a transgenic plant cell comprising a heterologous nucleic acid sequence operably linked to a promoter, wherein the heterologous nucleic acid sequence hybridizes to SEQ ID NO:1 under stringent hybridization conditions. Also provided is a transfected cell comprising a nucleic acid encoding an auxin-binding polypeptide and a non-naturally occurring nucleic acid sequence, wherein the heterologous nucleic acid sequence hybridizes to SEQ ID NO:1 or under stringent hybridization conditions, and, the auxin-binding polypeptide, upon expression in a plant cell, is capable of binding to plant auxin.

The invention also provides transgenic plants comprising a heterologous nucleic acid sequence operably linked to a promoter, wherein the heterologous nucleic acid sequence hybridizes to SEQ ID NO:1 under stringent hybridization conditions. In one embodiment, the promoter is a constitutive promoter, where the nucleic acid (and the ABP polypeptide) is constitutively expressed. In other embodiments, the promoter is a tissue- or cell-specific promoter, which can be primarily active in cotton fibers. Alternatively the promoter can be developmentally regulated, such as, e.g., primarily active in late primary and early secondary wall synthesis stages. The invention also provides transgenic plants, or progeny thereof, into which a heterologous nucleic acid sequence which hybridizes to SEQ ID NO:1 under stringent hybridization conditions has been introduced, wherein the nucleic acid encodes an ABP. Thus, the ABP, upon expression in a plant cell, is capable of binding to plant auxin. In one embodiment, these stringent hybridization conditions can comprise a hybridization step having a salt concentration of about 0.02 molar at pH 7 and a temperature of about 60° C. In the transgenic cells and plants of the invention, the heterologous nucleic acid can have a sequence as set forth in SEQ ID NO:1, or fragments thereof. The ABP-encoding nucleic acids of the invention are inserted into all plants with fiber cells influenced by auxin levels, including, e.g., cotton, silk cotton tree (Kapok, Ceiba pentandra), desert willow, creosote bush, winterfat, balsa, ramie, kenaf, hemp, roselle, jute, sisal abaca and flax. In alternative embodiments, the transgenic plants of the invention can be members of the genus Gossypium, including members of any Gossypium species, such as G. arboreum;. G. herbaceum, G. barbadense, and G. hirsutum.

The invention further provides methods for generating antibodies reactive with the auxin binding proteins (ABPs) of the invention. In one embodiment, the method comprises administering an immunogenically effective amount of ABP polypeptide encoded by a nucleic acid of the invention to a mammal. In another embodiment, the method comprises administering a nucleic acid, an expression cassette or vector comprising an ABP nucleic acid sequence of the invention to a mammal, wherein the ABP polypeptide is expressed in vivo to generate an anti-ABP antibody. An anti-ABP antibody can also be recombinantly produced in a transformed plant cell or a transgenic plant (as described below). In an alternative embodiment, the antibody is generated by screening a recombinant nucleic acid expression library, such as a phage (antibody) display library, for the expression of antigen binding sites (e.g., antibodies or antigen binding fragments) capable of binding to any portion of an ABP polypeptide of the invention, or fragment thereof.

The invention also provides a method for detecting an ABP nucleic acid in a nucleic acid-containing biological sample. In one embodiment, the method comprises the following steps: (a) contacting the sample with an ABP nucleic acid of the invention, (b) hybridizing the ABP nucleic acid to the nucleic acid in the sample; and, (c) detecting hybridization of the nucleic acids. The biological sample can comprise a transformed plant cell or a transgenic plant, such as, e.g., cotton plants (or any other fiber expressing plant, as described herein), particularly, members of the Gossypium species G. arboreum;. G. herbaceum, G. barbadense, and G. hirsutum. In alternative embodiments of this method, the ABP nucleic acid is an oligonucleotide primer pair capable of amplifying a subsequence of an ABP nucleotide of the invention, and detecting the hybridization of the ABP and sample nucleic acids comprises detection of an amplification product.

A further understanding of the nature and advantages of the present invention may be realized by reference to the remaining portions of the specification and the claims.

All publications, GenBank Accession references (sequences), patents and patent applications cited herein are hereby expressly incorporated by reference for all purposes.

### DETAILED DESCRIPTION OF THE INVENTION

This invention provides genetically engineered cells and plants with altered responses to auxin. In particular, the invention provides a family of novel auxin-binding polypeptide (ABP)-encoding nucleic acids. For example, the nucleic acids can be used to produce genetically engineered cotton cells and plants with improved fiber properties. The improved fibers, i.e., longer fibers, are the result of the intracellular expression of these recombinant ABPs.

While the invention is not limited by any particular mechanism of action, it is believed that the rate and duration of fiber expansion is under the ultimate control of auxin (defined below). The unique distribution of in vivo expressed auxin-binding polypeptides (ABPs) at sites coinciding with the deposition of newly synthesized cell wall material suggests that ABPs are generating their fiber-enhancing effect by playing a key role in the auxin-mediated expansion of developing fibers. One means by which the ABPs of the invention generate this improvement in fiber quality is by their ability to bind auxin. Enhanced levels of ABPs (e.g., generated by intracellular expression of recombinant ABPs of the invention) can shift the release of auxin from conjugated, inactive to active pools inside the cell or plant (as related to the rate of fiber expansion and timing of the growth of the fiber cells). Thus, an increase in ABP, by binding auxin, effectively increases the amounts of active, “fiber-enhancing,” auxin in the cell.

The ABP nucleic acids of the invention are used to generate transgenic plants capable of generating improved plant fiber. The transduced cells and transgenic plants of the invention include the source of the most commercially useful plant fiber, cotton, including Gossypium arboreum, Gossypium herbaceum, Gossypium barbadense and Gossypium hirsutum. The ABPs of the invention are also useful in generating improved fibers from other fiber-producing plants, e.g., silk cotton tree (Kapok, Ceiba pentandra), desert willow, creosote bush, winterfat, balsa, ramie, kenaf, hemp, roselle, jute, sisal abaca and flax, to name just a few.

Definitions

To facilitate understanding the invention, and to provide additional guidance to one of skill in the practice of the invention, a number of terms are defined below. Unless defined otherwise, all technical and scientific terms used herein have the meaning commonly understood by a person skilled in the art to which this invention belongs.

The term “amplifying” as used herein incorporates its common usage and refers to the use of any suitable amplification methodology for generating or detecting recombinant or naturally expressed nucleic acid, as described in detail, below. For example, the invention provides methods and reagents (e.g., oligonucleotide PCR primer pairs) for amplifying (e.g., by PCR) natually expressed or recombinant auxin binding protein (ABP)-encoding nucleic acids of the invention in vivo or in vitro.

As used herein, the term “auxin” refers to a class of phytohormone or plant growth regulators that control cell expansion. Auxins include indole-3-acetic acid, indoleacetic acid, or, IAA; see, e.g., Bennett (1998) Philos. Trans. R. Soc. Lond. B. Biol. Sci. 353:1511-1515; Guilfoyle (1998) Plant Physiol. 118(2):341-347, for further details on the structure and physiology of auxins.

A “ABP polynucleotide” is a nucleic acid sequence comprising (or consisting of) a coding region of about 50 to about 750 nucleotides, sometimes from about 100 to about 600 nucleotides and sometimes from about 300 to about 500 nucleotides, which hybridizes to SEQ ID NO:1 under stringent conditions (as defined below), or which encodes ABP polypeptide or fragment of at least 15 amino acids thereof.

As used herein, the term “operably linked,” refers to a functional relationship between two or more nucleic acid (e.g., DNA) segments. Typically, it refers to the functional relationship of a transcriptional regulatory sequence to a transcribed sequence. For example, a cis-acting transcriptional control element (a promoter) is operably linked to a coding sequence if it stimulates or modulates the transcription of the coding sequence in an appropriate host cell or other (e.g., in vitro) expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance i.e., they are trans-acting.

The term “promoter” refers to a region or sequence determinants located upstream or downstream from the start of transcription (they are cis-acting) and which are involved in recognition and binding of RNA polymerase and/or other proteins to initiate (or help initiate) transcription. A “plant promoter” is a promoter capable of initiating and/or regulating transcription in plant cells; see also discussion on plant promoters.

The term “constitutive promoter” refers to a promoter that initiates and helps control transcription in all cells and tissues. Promoters that drive expression continuously under physiological conditions are referred to herein as “constitutive” promoters and are active under most environmental conditions and states of development or cell differentiation; see also detailed discussion.

The term “inducible promoter” refers to a promoter which directs transcription under the influence of changing environmental conditions. Examples of environmental conditions that may effect transcription by inducible promoters include anaerobic conditions, elevated temperature, drought, or the presence of light. Such promoters are referred to herein as “inducible” promoters; see also detailed discussion.

The term “tissue-specific promoter” refers to a class of transcriptional control elements that are only active in particular cells or tissues. Examples of plant promoters that are only active in certain cell or tissue types include promoters that initiate transcription only (or primarily only) in certain tissues, such as, e.g., fibers (e.g., cotton fibers), roots, leaves, fruit, ovules, seeds, pollen, pistils, or flowers; see also detailed discussion. For example, as used herein, the term “primarily active in cotton fibers” means a transcriptional control element is primarily active in cotton fiber cells.

The term “developmentally regulated promoter” refers to a class of transcriptional control elements that are only active at particular stages of development, for example the stage of rapid elongation in a cotton fiber cell. For example, as used herein, the term “primarily active in late primary and early secondary wall synthesis stages” means a transcriptional control element is primarily active only at this stage of development.

The term “plant” includes whole plants, plant organs (e.g., leaves, stems, flowers, roots, etc.), plant protoplasts, seeds and plant cells and progeny of same. The class of plants which can be used in the method of the invention is generally as broad as the class of higher plants amenable to transformation techniques, including angiosperms (monocotyledonous and dicotyledonous plants), as well as gymnosperms. It includes plants of a variety of ploidy levels, including polyploid, diploid, haploid and hemizygous (see detailed discussion).

The term “antibody” refers to a peptide or polypeptide substantially encoded by an immunoglobulin gene or immunoglobulin genes, or fragments thereof, capable of specifically binding an epitope, as contained in an auxin binding polypeptide of the invention; see, e.g. Fundamental Immunology, Third Edition, W. E. Paul, ed., Raven Press, N.Y. (1993); Wilson (1994) J. Immunol. Methods 175:267-73; Yarmush (1992) J. Biochem. Biophys. Methods 25:85-97. One of skill will appreciate that antibody fragments may be isolated or synthesized de novo either chemically or by utilizing recombinant DNA methodology. The term antibody also includes “chimeric” antibodies either produced by the modification of whole antibodies or those synthesized de novo using recombinant DNA methodologies. Immunoglobulins can also be generated using phage display libraries, and variations thereof, as described below.

The term “expression cassette” refers to any recombinant expression system for the purpose of expressing a nucleic acid sequence of the invention in vitro or in vivo, constitutively or inducibly, in any cell, including prokaryotic, yeast, fungal, plant, insect or mammalian cell. The term includes linear or circular expression systems. The term includes expression cassettes, e.g., vectors, that remain episomal or integrate into the host cell genome. The expression cassettes can have the ability to self-replicate or not, i.e., drive only transient expression in a cell. The term includes recombinant expression cassettes which contain only the minimum elements needed for transcription of the recombinant nucleic acid.

As used herein, “isolated,” when referring to a molecule or composition, such as, for example, an auxin binding polypeptide or a nucleic acid encoding this polypeptide, means that the molecule or composition is separated from at least one other compound, such as a protein, other nucleic acids (e.g., RNAs), or other contaminants with which it is associated in vivo or in its naturally occurring state. Thus, an auxin binding polypeptide or nucleic acid is considered isolated when it has been isolated from any other component with which it is naturally associated, e.g., cell membrane, as in a cell extract. An isolated composition can, however, also be substantially pure. An isolated composition can be in a homogeneous state and can be in a dry or an aqueous solution. Purity and homogeneity can be determined, e.g., using analytical chemistry techniques such as polyacrylamide gel electrophoresis (SDS-PAGE), high performance liquid chromatography (HPLC) and NMR spectroscopy.

The term “nucleic acid molecule” or “nucleic acid sequence” refers to a deoxyribonucleotide or ribonucleotide oligonucleotide in either single- or double-stranded form. The term encompasses nucleic acids, i.e., oligonucleotides, containing known analogues of natural nucleotides which have similar or improved binding properties as the deoxyribonucleotide or ribonucleotide nucleic acids of the invention. The term also encompasses nucleic-acid-like structures with synthetic backbones. DNA backbone analogues provided by the invention include, e.g., phosphodiester, phosphorothioate, phosphorodithioate, methyl-phosphonate, phosphoramidate, alkyl phosphotriester, sulfamate, 3′-thioacetal, methylene (methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described in WO 97/03211; WO 96/39154; Mata (1997) Toxicol Appl Pharmacol 144:189-197. Other synthetic backbones encompasses by the term include e.g., methyl-phosphonate linkages or alternating methylphosphonate and phosphodiester linkages (Strauss-Soukup (1997) Biochemistry 36:8692-8698), and benzylphosphonate linkages (Samstag (1996) Antisense Nucleic Acid Drug Dev 6:153-156). The term nucleic acid is used interchangeably with gene, cDNA, mRNA, oligonucleotide primer, probe and amplification product.

As used herein, the “sequence” of a gene (unless specifically stated otherwise) or nucleic acid refers to the order of nucleotides in the polynucleotide, including either or both strands of a double-stranded DNA molecule, e.g., the sequence of both the coding strand and its complement, or of a single-stranded nucleic acid molecule. For example, exemplary ABP-encoding nucleic acids of the invention have sequences as set forth in SEQ ID NO:1.

The terms “heterologous nucleic acid” and “exogenous nucleic acid” refer to a nucleic acid that has been isolated, synthesized, cloned, ligated, excised in conjunction with another nucleic acid, in a manner that is not found in nature, and/or introduced into and/or expressed in a cell or cellular environment other than or at levels or forms different than the cell or cellular environment in which said nucleic acid or protein is be found in nature. The term encompasses nucleic acids originally obtained from a different organism, plant, cell type or cell line than that in which it is naturally expressed.

The term “recombinant,” when used with reference to a cell, or to a nucleic acid (or expression cassette or vector), polypeptide or peptide, refers to a material, or a material corresponding to the natural or native form of the material, that has been modified by the introduction of a new moiety or alteration of an existing moiety (i.e., nucleotide or amino acid residues), or is identical thereto but produced or derived from synthetic materials. For example, recombinant cells express genes that are not found within the native (non-recombinant) form of the cell or express native genes that are otherwise expressed at a different level (typically, under-expressed or over-expressed). The term “recombinant means” encompasses all means of expressing, i.e., transcription or translation of, an isolated and/or cloned nucleic acid or polypeptide in vitro or in vivo. For example, the term “recombinant means” encompasses techniques where a recombinant nucleic acid, such as a cDNA encoding a protein, is inserted into an expression system (expression cassettee or vector), which is introduced into a cell and the cell expresses the protein. “Recombinant means” also encompass the ligation of nucleic acids having coding or promoter sequences from different sources into an expression cassettee or vector for expression of a fusion protein; or, inducible, constitutive, tissue-specific or developmentally controlled expression of a protein, such as the ABPs of the invention.

The term “specifically hybridizes” refers to a nucleic acid that hybridizes, duplexes or binds to a particular target DNA or RNA sequence. The target sequences can be present in a preparation of total cellular DNA or RNA. Proper annealing conditions depend, e.g., upon a nucleic acid's characteristics, such as a length, base composition, and the number of mismatches between the probe and target. Appropriate conditions to achieve a desired result (e.g., stringent conditions for stringent hybridization) can be readily determined empirically using routine screening of reagents, conditions, etc. (see below). For discussions of nucleic acid “probe” design and annealing conditions, see, e.g., see e.g., Sambrook, ed., MOLECULAR CLONING: A LABORATORY MANUAL (2ND ED.), Vols. 1-3, Cold Spring Harbor Laboratory, (1989) (“Sambrook”); CURRENT PROTOCOLS IN MOLECULAR BIOLOGY, Ausubel, ed. John Wiley & Sons, Inc., New York (1997) (“Ausubel”); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993) (“Tijssen”).

The terms “stringent hybridization,” “stringent conditions,” or “specific hybridization conditions” refers to conditions under which a polynucleotide (e.g., an oligonucleotide), when used, e.g., as a probe or primer, will primarily hybridize to its target sequence (which may be a subsequence of a larger molecule), such as an ABP-encoding nucleic acid of the invention, but will not bind in significant amounts to any other unrelated sequence. Stringent conditions are sequence-dependent.

Longer sequences hybridize specifically at higher temperatures. Stringent conditions are selected to be about 5° C. lower than the thermal melting point (Tₘ) for the specific sequence at a defined ionic strength and pH. The Tₘ is the temperature (under defined ionic strength, pH, and nucleic acid concentration) at which 50% of the probes complementary to the target sequence hybridize to the target sequence at equilibrium (if the target sequences are present in excess, at Tₘ, 50% of the probes are occupied at equilibrium). Typically, stringent conditions will be those in which the hybridization conditions have a salt concentration that is less than about 1.0 M sodium ion, i.e., about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). For example, in a preferred embodiment of the invention, the stringent hybridization conditions comprise a hybridization step having a salt concentration of about 0.02 molar at pH 7 and a temperature of about 60° C. Stringent conditions may also be achieved with the addition of destabilizing agents, such as formamide, to the hybridization solution. Often, high stringency wash conditions preceded by low stringency wash conditions are used to remove possible background probe signals. An example of useful wash conditions for a duplex of, e.g., more than 100 nucleotides, is 0.2× SSC for at 45° C., 50° C., or 60° C. for 20 minutes (see, e.g., Sambrook for a description of SSC buffer). An example of lower stringency wash for a duplex of, e.g., more than 100 nucleotides, is 4-6× SSC at 40° C. for 15 minutes. A signal to noise ratio of 2x (or higher) than that observed for an unrelated probe in a hybridization assay indicates detection of a “specific hybridization.” Nucleic acids which do not hybridize to each other under stringent conditions can still be substantially identical if the polypeptides which they encode are substantially identical. This can occur, e.g., when a nucleic acid is created that encodes conservative substitutions. “Stringent” parameters (including both hybridization and wash conditions) are different under different environments (depending on the methodology), e.g., as in the different “stringent” parameters used in Southern versus Northern hybridizations.

In the case of both expression of transgenes and inhibition of endogenous genes (e.g., by antisense, or sense suppression) one of skill will recognize that the inserted polynucleotide sequence need not be identical and may be “substantially identical” to a sequence of the gene from which it was derived. For example, in the case of polynucleotides used to inhibit expression of an endogenous gene, the introduced sequence need not be perfectly identical to a sequence of the target endogenous gene. The introduced polynucleotide sequence will typically be at least substantially identical (as determined below) to the target endogenous sequence. Similarly, in the case where the inserted polynucleotide sequence is transcribed and translated to produce a functional polypeptide, one of skill will recognize that because of codon degeneracy a number of polynucleotide sequences will encode the same polypeptide. These variants are specifically covered by the term “polynucleotide sequence from” an ABP gene of the invention. In addition, the invention specifically includes sequences (e.g., full length sequences) substantially identical (determined as described below) with a ABP sequence exemplified here and that encode proteins that retain the function of an ABP polypeptide.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

Optimal alignment of sequences for comparison may be conducted by the local homology algorithm of Smith and Waterman Add. APL. Math. 2:482 (1981), by the homology alignment algorithm of Needle man and Wunsch J Mol. Biol. 48:443 (1970), by the search for similarity method of Pearson and Lipman Proc. Natl. Acad. Sci. (U.S.A.) 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection. “Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 25% sequence identity. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 90%, 95%, or 99%. compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%. Preferred percent identity of polypeptides can be any integer from 40% to 100%. More preferred embodiments include at least 40%, 45%, 50%, 55%, 60%, 65%, 70%, 75%, 80%, 85%, 90%, 95%, or 99%. Most preferred embodiments include 61%, 62%, 63%, 64%, 65%, 66%, 67%, 68%, 69%, 70%, 71%, 72%, 73%, 74% and 75%. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

As used herein, the term “transgenic plant” means a plant into which an exogenous (i.e., heterologous) nucleic acid sequence has been inserted. The exogenous nucleic acid can be inserted into vegetative tissue, in which case the transgenic plant will not produce progeny with the transduced nucleic acid. Alternatively, the exogenous nucleic acid can be stably integrated into germline tissue and will be passed on to the plant's progeny. Research with transgenic plants has demonstrated that they are capable of passing on the inserted genes to their progeny by normal Mendelian inheritance (see, e.g., Christou (1990) Trends in Biotechnol. 8:145-151). Thus, both vegetative transgenic and germ-line transgenic plants (which inherit the inserted genetic construct)are transgenic plants of the invention.

General Techniques

The auxin-binding polypeptide (ABP)-encoding nucleic acid sequences of the invention, whether RNA, cDNA, genomic DNA, or hybrids thereof, may be isolated from a variety of sources, genetically engineered, amplified, and/or expressed recombinantly. Alternatively, these nucleic acids can be chemically synthesized in vitro. Techniques for the manipulation of nucleic acids, such as, e.g., subcloning into expression vectors, labeling probes, sequencing, and hybridization under different conditions (e.g., stringent conditions) are well described in the scientific and patent literature, see e.g., Sambrook, Ausubel, Tijssen.

Isolation, Synthesis, and Purification of Nucleic Acids Encoding ABPs

The invention provides for nucleic acids encoding a polypeptide capable of binding to auxin, where the nucleic acid sequence hybridizes to SEQ ID NO:1 under defined stringent hybridization conditions (as defined above). Such a sequence is generally more than 65% identical to the nucleic acid shown in SEQ ID NO:1, frequently more than 75% identical and often more than 85% identical. Thus, the invention provides ABP-encoding mRNA, cDNA and genes, which may be obtained or identified using primers and nucleic acid probes capable of hybridizing to SEQ ID NO:1 under various conditions (depending, e.g., on the target sequence of the probes, e.g., as targetting the 5′ nonconserved region of the APB, see below for further discussion):

An exemplary ABP-encoding nucleic acid of the invention from cotton has a sequence as set forth in SEQ ID NO:1. Alternatively, the ABP subregion outside of the unique 5′ region are preferred for identifying further ABP species. The ABP nucleic acids so discovered can be sequenced and compared to the exemplary ABP of the invention (SEQ ID NO:1) for sequence identity. These newly identified ABP nucleic acids can be functionally assessed by, e.g., generation of their corresponding ABP polypeptides, and determining their ability to bind auxin, or, when expressed in vivo, to effect the rate or extent of growth of fiber cells, as described below.

Additional ABP sequences can also be identified and characterized using various methods, including: i) computer searches of DNA databases for DNAs containing sequences conserved with ABP genes and having sequence identity with conserved ABP polypeptide structural domains (motifs) described above (i.e., the subspecies unique 5′ region), ii) hybridization with a probe from a known ABP gene sequence to mRNA, cDNA or DNA sequence or libraries from a target plant, and, iii) by PCR or other signal or target amplification technologies using primers complementary to regions highly conserved (shared) amongst different ABP subspecies (e.g., their structurally similar domains, as the region outside of the 5′-nonconserved domain).

Nucleic acid amplification methods, such as PCR, are illustrated as an exemplary means used to identify, isolate and generate members of the ABP genus of the invention. Amino acid sequences can be conserved, but, because of the degeneracy of the genetic code, codon usage bias, or amino acid changes, the DNA sequences corresponding to conserved polypeptide structural domain (motif) regions (i.e., the region outside of the 5′-non-conserved domain) can be different between different plants. For this reason, one can employ in the methods nucleotides at the positions in the primers that are degenerate for a particular amino acid to ensure that one or more of the different primers can hybridize to an ABP species (or subspecies) whose nucleotide sequence is not completely known. In performing amplification with such primers, one may take allowances for the degenerate positions probe by using conditions appropriate for allowing certain base mismatches to occur, e.g., in hybridization or in the annealing steps of PCR, i.e., degenerate PCR conditions. Primers for identifying any member of the genus of ABP genes and polypeptides are encompassed by the invention. For example, the skilled artisan, using the sequences set forth herein and a degenerate PCR technique, can design and use such amplification primers to identify additional ABP nucleotides and polypeptides.

Nucleic Acid Hybridization Techniques

The hybridization techniques disclosed herein can be utilized to identify, isolate and characterize genes and gene products (i.e., mRNA) encoding for the ABP species of the invention. For example, a nucleic acid sequence of the invention can be identified by its ability to hybridize to SEQ ID NO:1 under stringent hybridization conditions, its ability to bind auxin, its ability, when expressed in a plant cell, to generate a polypeptide able to effect the cell's auxin-mediated rate or extent of growth.

A variety of methods for specific DNA and RNA detection and measurement using nucleic acid hybridization techniques are known to those of skill in the art. See, e.g., NUCLEIC ACID HYBRIDIZATION, A PRACTICAL APPROACH, Ed. Hames et al., IRL Press, 1985; Gall (1989) Proc. Natl. Acad. Sci. USA 63:378; Sambrook, and the like. Another means for determining the level of expression of a gene encoding a protein is in situ hybridization. In situ hybridization assays are well known and are generally described in Angerer (1987) Methods Enzymol 152:649; for use in plant, see, e.g., Klinge (1997) Mol. Gen. Genet. 255:248-257; Bonhomme (1997) Plant Mol. Biol. 34:573-582; Piffanelli (1997) Plant J. 11: 549-562. Another well-known in situ hybridization technique is the so-called FISH fluorescence in situ hybridization, as described, e.g., by Macechko (1997) J. Histochem. Cytochem. 45:359-363; Ji (1997) Genome 40:34-40; Raap (1995) Hum. Mol. Genet. 4:529-534.

Amplification of Nucleic Acids Encoding ABP Polypeptides

The present invention provides oligonucleotide primers and probes that can hybridize specifically to and amplify nucleic acids having ABP protein-encoding (cDNA) or genomic nucleic acid, such as the exemplary ABP species with sequences as set forth in SEQ ID NO:1. Such reagents can be used to identify all ABP protein-encoding and genomic sequences. Included in the invention's genomic sequences are intronic and genomic, non-transcribed sequences, promoters, and enhancers which can also be amplified using the PCR primers of the invention (including degenerate or other primers, as in RACE) to identify new ABP species.

Amplification of ABP sequences which are conserved amongst different members of the genus, i.e., structurally conserved sequences, such as the region outside of the 5′ subspecie unique subsequence, generate oligonucleotides that are preferred reagents for such amplifications. These reagents are also used as hybridization probes to identify and isolate additional ABP species from other plants. These oligonucleotides can also be used as primers to directly amplify additional species, using any amplification technique, such as, for example RACE (see, e.g., Lankiewicz (1997) Nucleic Acids Res 25:2037-2038; Frohman (1988) Proc. Natl. Acad. Sci. USA 85:8998; Doenecke (1997) Leukemia 11:1787-1792).

Oligonucleotides can be used to identify and detect additional ABP species using a variety of hybridization techniques and conditions. Suitable amplification methods include, but are not limited to: polymerase chain reaction, PCR (PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y. (Innis)), ligase chain reaction (LCR) (Wu (1989) Genomics 4:560; Landegren (1988) Science 241:1077; Barringer (1990) Gene 89:117); transcription amplification (Kwoh Proc. Natl. Acad. Sci. USA, 86:1173 (1989)); and, self-sustained sequence replication (Guatelli (1990) Proc. Natl. Acad. Sci. USA, 87:1874); Q Beta replicase amplification (Smith (1997) J. Clin. Microbiol. 35:1477-1491, automated Q-beta replicase amplification assay; Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316, Sambrook, and Ausubel, as well as Mullis (1987) U.S. Pat, Nos. 4,683,195 and 4,683,202; Arnheim (1990) C&EN 36-47; Lomell J. Clin. Chem., 35:1826 (1989); Van Brunt (1990) Biotechnology, 8:291-294; Wu (1989) Gene 4:560; Sooknanan (1995) Biotechnology 13:563-564. Methods for cloning in vitro amplified nucleic acids are described in Wallace, U.S. Pat. No. 5,426,039. See also, Wassenegger (1995), “Application of PCR to transgenic plants,” Methods Mol Biol 49:423-37; Garvey (1991) Biotechniques 11:428-32.

PCR-amplified sequences can also be labeled and used as detectable oligonucleotide probes, but such nucleic acid probes can be generated using any synthetic or other technique well known in the art. The labeled amplified DNA or other oligonucleotide or nucleic acid of the invention can be used as probes to further identify and isolate ABP species from various cDNA or genomic libraries.

Alignment Analysis of ABP Gene Sequences

The genus of ABP nucleic acid sequences of the invention includes genes and gene products identified and characterized by analysis using the sequences nucleic acid and protein sequences of the invention, including SEQ ID NO:1 and SEQ ID NO:2. Optimal alignment of sequences for comparison can use any means to analyze sequence identity (homology) known in the art, e.g., by the progressive alignment method of termed “PILEUP” (see below); by the local homology algorithm of Smith & Waterman (1981) Adv. Appl. Math. 2: 482; by the homology alignment algorithm of Needleman & Wunsch (1970) J. Mol. Biol. 48:443; by the search for similarity method of Pearson (1988) Proc. Natl. Acad. Sci. USA 85: 2444; by computerized implementations of these algorithms (e.g., GAP, BESTFIT, FASTA, and TFASTA in the Wisconsin Genetics Software Package, Genetics Computer Group, 575 Science Dr., Madison, Wis.); ClustalW (CLUSTAL in the PC/Gene program by Intelligenetics, Mountain View, Calif., described by, e.g., Higgins (1988) Gene 73: 237-244; Corpet (1988) Nucleic Acids Res. 16:10881-90; Huang (1992) Computer Applications in the Biosciences 8:155-65, and Pearson (1994) Methods in Molec. Biol. 24:307-31), Pfam (Sonnhammer (1998) Nucleic Acids Res. 26:322-325); TreeAlign (Hein (1994) Methods Mol. Biol. 25:349-364; MES-ALIGN, and SAM sequence alignment computer programs; or, by inspection. See also Morrison (1997) Mol. Biol. Evol. 14:428-441, as an example of the use of PILEUP.

Another example of algorithm that is suitable for determining sequence similarity is the BLAST algorithm, which is described in Altschul (1990) J. Mol. Biol. 215: 403-410. Software for performing BLAST analyses is publicly available through the National Center for Biotechnology Information, http://www.ncbi.nlm.nih.gov/; see also Zhang (1997) Genome Res. 7:649-656 (1997) for the “PowerBLAST” variation. This algorithm involves first identifying high scoring sequence pairs (HSPs) by identifying short words of length W in the query sequence that either match or satisfy some positive-valued threshold score T when aligned with a word of the same length in a database sequence. T is referred to as the neighborhood word score threshold (Altschul et al, supra.). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a wordlength (W) of 11, the BLOSUM62 scoring matrix (see Henikoff (1992) Proc. Natl. Acad. Sci. USA 89: 10915-10919) alignments (B) of 50, expectation (E) of 10, M=5, N=−4, and a comparison of both strands. The term BLAST refers to the BLAST algorithm which performs a statistical analysis of the similarity between two sequences; see, e.g., Karlin (1993) Proc. Natl. Acad. Sci. USA 90:5873-5787. One measure of similarity provided by the BLAST algorithm is the smallest sum probability (P(N)), which provides an indication of the probability by which a match between two nucleotide or amino acid sequences would occur by chance.

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence (i.e., a nucleic acid would be considered an ABP nucleic acid of the invention) if the smallest sum probability in a comparison of the test nucleic acid to the reference nucleic acid is less than about 0.1, more preferably less than about 0.01, and most preferably less than about 0.001.

Expression of Recombinant ABP Polypeptides

The invention provides for methods and reagents for the expression of the ABP nucleic acids of the invention in any prokaryotic, eukaryotic, yeast, fungal, plant, insect, or animal cell. Any recombinant expression system can be used; as for expression in plants to generate fibers of improved strength and length; or, in expression systems for generating large amounts of ABP polypeptide for structural analyses, or as antigen for generating antibodies, including, e.g., bacterial, yeast, insect or mammalian systems. The ABP-expressing nucleic acids of the invention may be introduced into a genome or into the cytoplasm or a nucleus of a cell and expressed by a variety of conventional techniques, well described in the scientific and patent literature. See, for example Roberts (1987) Nature 328:731; Berger (1987) supra; Schneider (1995) Protein Expr. Purif. 6435:10; Sambrook and Ausubel. The transcriptional regulatory elements (e.g., promoters, enhancers), expression cassettes, vectors, makers, fusion protein elements, and other functional elements needed to practice the invention can be isolated from natural sources, obtained from such sources as ATCC or GenBank libraries, or prepared by synthetic or recombinant methods, as described herein. A few selected illustrative general and specific teaching examples relevant to such technology are described below.

Vectors and Transcriptional Control Elements

The invention, providing methods and reagents for making the novel genus of ABP nucleic acids described herein, further provides methods and reagents for expressing these nucleic acids using novel expression cassettes and vectors in transformed cells and transgenic plants. Constitutive and inducible transcriptional and translational cis- (e.g., promoters and enhancers) and trans-acting control elements are incorporated in the constructs, transformed cells and transgenic plants of the invention.

The expression of natural, recombinant or synthetic ABP polypeptide-encoding or other (e.g., antisense, ribozyme) nucleic acids can be achieved by operably linking the coding region a promoter (that can be plant-specific or not, constitutive or inducible), incorporating the construct into expression cassettes or vectors, and introducing the resultant construct into an in vitro system or a suitable host cell or plant. Synthetic procedures may also be used. Typical expression systems contain, in addition to coding or antisense sequence, transcription and translation terminators, polyadenylation sequences, transcription and translation initiation sequences, and promoters useful for transcribing DNA into RNA. The expression systems optionally at least one independent terminator sequence, sequences permitting replication of the cassette in vivo, e.g., plants, eukaryotes, or prokaryotes, or a combination thereof, (e.g., shuttle vectors) and selection markers for the selected expression system, e.g., plant, prokaryotic or eukaryotic systems. To ensure proper polypeptide expression under varying conditions, a polyadenylation region at the 3′-end of the coding region can be included; see Li (1997) Plant Physiol. 115:321-325, for a review of the polyadenylation of RNA in plants. The polyadenylation region can be derived from the natural gene, from a variety of other plant genes, or from T-DNA (e.g., using Agrobacterium tumefaciens T-DNA replacement vectors, see e.g., Thykjaer (1997) Plant Mol Biol. 35:523-530; using a plasmid containing a gene of interest flanked by Agrobacterium T-DNA border repeat sequences; Hansen (1997) “T-strand integration in maize protoplasts after codelivery of a T-DNA substrate and virulence genes,” Proc. Natl. Acad. Sci. USA 94:11726-11730.

The nucleic acids of the invention can be expressed in expression cassettes, vectors or viruses which are transiently expressed in cells using, for example, episomal expression systems (e.g., cauliflower mosaic virus (CaMV) viral RNA is generated in the nucleus by transcription of an episomal minichromosome containing supercoiled DNA, Covey (1990) Proc. Natl. Acad. Sci. USA 87:1633-1637). Expression vectors capable of expressing proteins in plants are well known in the art, and can include, e.g., vectors from Agrobacterium spp., potato virus X (see, e.g. Angell (1997) EMBO J. 16:3675-3684), tobacco mosaic virus (see, e.g., Casper (1996) Gene 173:69-73), tomato bushy stunt virus (see, e.g., Hillman (1989) Virology 169:42-50), tobacco etch virus (see, e.g., Dolja (1997) Virology 234:243-252), bean golden mosaic virus (see, e.g., Morinaga (1993) Microbiol Immunol. 37:471-476), cauliflower mosaic virus (see, e.g., Cecchini (1997) Mol. Plant Microbe Interact. 10:1094-1101), maize Ac/Ds transposable element (see, e.g., Rubin (1997) Mol. Cell. Biol. 17:6294-6302; Kunze (1996) Curr. Top. Microbiol. Immunol. 204:161-194), and the maize Suppressor-mutator (Spm) transposable element (see, e.g., Schlappi (1996) Plant Mol. Biol. 32:717-725); and derivatives thereof. Alternatively, coding sequences, i.e., all or subfragments of SEQ ID NO:1 (e.g., those encoding biologically acitive fragments of SEQ ID NO:2), can be inserted into the host cell genome becoming an integral part of the host chromosomal DNA.

Selection markers can be incorporated into expression cassettes and vectors. These can confer a selectable phenotype on transformed cells. Sequences coding for episomal maintenance and replication are used if integration into the host genome is not desired. For example, the marker may encode antibiotic resistance, particularly resistance to chloramphenicol, kanamycin, G418, bleomycin, hygromycin, or herbicide resistance, such as resistance to chlorosulfuron or Basta, to permit selection of those cells transformed with the desired DNA sequences, see e.g., Blondelet-Rouault (1997) Gene 190:315-317; Aubrecht (1997) J. Pharmacol. Exp. Ther. 281:992-997. See also, Mengiste (1997) Plant J. 12:945-948, showing that the 1′ promoter is an attractive alternative to the cauliflower mosaic virus (CaMV) 35S promoter for the generation of T-DNA insertion lines, the 1′ promoter may be especially beneficial for the secondary transformation of transgenic strains containing the 35S promoter to exclude homology-mediated gene silencing.

Constitutive Promoters

In construction of recombinant expression cassettes, vectors and transgenic plants of the invention, a promoter fragment can be employed to direct expression of the desired gene in all tissues of a plant or animal. Promoters that drive expression continuously under physiological conditions are referred to as “constitutive” promoters and are active under most environmental conditions and states of development or cell differentiation. Examples of constitutive promoters include those from viruses which infect plants, such as the cauliflower mosaic virus (CaMV) 35S transcription initiation region (see, e.g., Dagless (1997) Arch. Virol. 142:183-191); the 1′- or 2′-promoter derived from T-DNA of Agrobacterium tumafaciens (see, e.g., Mengiste (1997) supra; O'Grady (1995) Plant Mol. Biol. 29:99-108); the promoter of the tobacco mosaic virus; the promoter of Figwort mosaic virus (see, e.g., Maiti (1997) Transgenic Res. 6:143-156); actin promoters, such as the Arabidopsis actin gene promoter (see, e.g., Huang (1997) Plant Mol. Biol. 1997 33:125-139); alcohol dehydrogenase (Adh) gene promoters (see, e.g., Millar (1996) Plant Mol. Biol. 31:897-904); and, other transcription initiation regions from various plant genes known to those of skill. See also Holtorf (1995) “Comparison of different constitutive and inducible promoters for the overexpression of transgenes in Arabidopsis thaliana,” Plant Mol. Biol. 29:637-646.

Inducible Promoters

Alternatively, a plant promoter may direct expression of the ABP nucleic acid of the invention under the influence of changing environmental conditions or developmental conditions.

Examples of environmental conditions that may effect transcription by inducible promoters include anaerobic conditions, elevated temperature, drought, or the presence of light. Such promoters are referred to herein as “inducible” promoters. For example, the invention incorporates the drought-inducible promoter of maize (Busk (1997) supra); the cold, drought, and high salt inducible promoter from potato (Kirch (1997) Plant Mol. Biol. 33:897-909).

Alternatively, plant promoters which are inducible upon exposure to plant hormones, such as auxins, are used to express the ABP nucleic acids of the invention. For example, the invention can use the auxin-response elements E1 promoter fragment (AuxREs) in the soybean (Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) Mol. Plant Microbe Interact. 10:933-937); and, the promoter responsive to the stress hormone abscisic acid (Sheen (1996) Science 274:1900-1902).

The ABP nucleic acids of the invention can also be operably linked to plant promoters which are inducible upon exposure to chemicals reagents which can be applied to the plant, such as herbicides or antibiotics. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. Coding sequence can be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324). Using chemically- (e.g., hormone- or pesticide-) induced promoters, i.e., promoter responsive to a chemical which can be applied to the transgenic plant in the field, expression of a polypeptide of the invention can be induced at a particular stage of development of the plant. Thus, the invention also provides for transgenic plants containing an inducible gene encoding for ABP polypeptides whose host range is limited to target plant species, such as cotton crops, inducible at any stage of development of the crop.

Tissue-Specific and Developmentally-Specific Promoters

Tissue specific promoters are transcriptional control elements that are only active(or primarily active) in particular cells or tissues, such as fiber cells, roots, leaves, fruit, ovules, seeds, pollen, pistils, or flowers. In alternative embodiments, plant promoters which are active only in specific tissues or at specific times during plant development are used to express the ABP nucleic acids of the invention.

Cotton Fiber Specific Promoters

In one embodiment, the ABP nucleic acids are operably linked to a promoter active primarily only in cotton fiber cells. In a preferred embodiment, the ABP nucleic acids of the invention are operably linked to a promoter active primarily during the stages of cotton fiber cell elongation, e.g., as described by Rinehart (1996) supra. The ABP nucleic acids are operably linked to the Fbl2A gene promoter that is preferentially expressed in cotton fiber cells (Ibid). See also, John (1997) Proc. Natl. Acad. Sci. USA 89:5769-5773; John, et al., U.S. Pat. Nos. 5,608,148 and 5,602,321, describing cotton fiber-specific promoters and methods for the construction of transgenic cotton plants.

Additional promoters which are linked to genes found to be expressed preferentially in cotton fiber cells can also be identified and isolated for incorporation into the expression cassettes and vectors of the invention. They care also used to express ABP nucleic acids in a cotton fiber specific (or fiber-preferential) manner. As the coding sequences for these tissue specific genes have been characterized, identification and isolation of these cotton fiber specific promoters can be accomplished using standard genetic engineering techniques. For example, Shimizu (1997) Plant Cell Physiol. 38:375-378, found that both endo-1,4-beta-glucanase and expansin mRNA levels were high during cotton fiber cell elongation, but decreased when cell elongation ceased. Xyloglucan also decreased. The endo-1,3-beta-glucanase mRNA level was very low in the elongating cells, but increased gradually at the onset of secondary wall synthesis, accompanying the massive deposition of cellulose. Also, as discussed above, Song (1997) supra, found a cotton fiber-specific acyl-carrier protein in Gossypium hirsutum. Ma (1997) Biochim. Biophys. Acta 1344:111-114, found a cotton fiber-specific cDNA encoding a lipid transfer protein. See also John, U.S. Pat. No. 5,597,718, describing means to identify cotton fiber-specific genes by differential cDNA library screenings.

Other Tissue Specific Promoters

Root-specific promoters may also be used in some embodiments of the present invention. Examples of root-specific promoters include the promoter from the alcohol dehydrogenase gene (DeLisle et al. Int. Rev. Cytol. 123, 39-60 (1990)).

Further examples include, e.g., ovule-specific, embryo-specific, endosperm-specific, integument-specific, seed coat-specific, or some combination thereof. A leaf-specific promoter has been identified in maize, Busk (1997) Plant J. 11:1285-1295. The ORF13 promoter from Agrobacterium rhizogenes exhibits high activity in roots (Hansen (1997) supra). A maize pollen-specific promoter has been identified, Guerrero (1990) Mol. Gen. Genet. 224:161-168). A tomato promoter active during fruit ripening, senescence and abscission of leaves and, to a lesser extent, of flowers can be used (Blume (1997) Plant J. 12:731-746); or a pistil-specific promoter from the potato SK2 gene, encoding a pistil-specific basic endochitinase (Ficker (1997) Plant Mol. Biol. 35:425-431). The Blec4 gene from pea is active in epidermal tissue of vegetative and floral shoot apices of transgenic alfalfa, making it a useful tool to target the expression of foreign genes to the epidermal layer of actively growing shoots or fibers. Another tissue-specific plant promoter is the ovule-specific BEL1 gene (Reiser (1995) Cell 83:735-742, GenBank No. U39944). See also Klee, U.S. Pat. No. 5,589,583, describing a plant promoter region is capable of conferring high levels of transcription in meristematic tissue and/or rapidly dividing cells.

One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well. In another embodiment, a nucleic acid of the invention is expressed through a transposable element. This allows for constitutive, yet periodic and infrequent expression of the ABP polypeptide.

The invention also provides for use of tissue-specific (or constitutive) promoters derived from viruses which can include, e.g., the tobamovirus subgenomic promoter (Kumagai (1995) Proc. Natl. Acad. Sci. USA 92:1679-1683; the rice tungro bacilliform virus (RTBV), which replicates only in phloem cells in infected rice plants, with its promoter which drives strong phloem-specific reporter gene expression; the cassava vein mosaic virus (CVMV) promoter, with highest activity in vascular elements, in leaf mesophyll cells, and in root tips (Verdaguer (1996) Plant Mol. Biol. 31:1129-1139).

Production of Transformants and Transgenic Plants

The invention provides for a variety of in vivo systems expressing the ABP polypeptides of the invention, including transformed cells and transgenic plants. The polypeptides of the invention are expressed in, in addition to plant cells, a variety of additional expression (and plant cell) systems to generate large amounts of protein for, e.g., in vitro functional testing, such as screening for compounds (auxin derivatives) that bind to a ABP polypeptide of the invention, to generate antibodies, structural studies (i.e., crystallization), to generate sufficient protein to apply to a plant to in fiber cell growth, and the like.

There are several well-known methods of introducing nucleic acids into plants (including bacterial and other cells), a process often called “transforming,” any of which may be used in the methods of the present invention (see, e.g., Sambrook). Techniques for transforming a wide variety of animal and plant cells are well known and described in the technical and scientific literature. See, e.g., Weising, Ann. Rev. Genet. 22:421-477 (1988) for plant cells and Sambrook for animal and bacterial cells. Specific examples of methods of expressing the novel ABP proteins of the invention are described below.

The present invention also provides methods and reagents for recombinant, genetically engineered ABP genes in a variety of plant cell systems. For example, these can include fusion of the recipient cells with bacterial protoplasts containing DNA, use of DEAE dextran, polyethylene glycol precipitation (described in Paszkowski (1984) Embo J. 3:2717-2722), infection with viral vectors, and the like. In plants, the DNA construct may be introduced directly into the genomic DNA of the plant cell using techniques such as electroporation (described in Fromm (1985) Proc. Natl. Acad. Sci. USA 82:5824) and microinjection of plant cell protoplasts (Schnorf (1991) Transgenic Res. 1:23-30), or the DNA constructs can be introduced directly to plant tissue using ballistic methods, such as DNA particle bombardment (discussed further below), or DNA can be introduced using viruses.

Plant cells can be transformed using viral vectors, such as, e.g., tobacco mosaic virus derived vectors (Rouwendal (1997) Plant Mol. Biol. 33:989-999), see Porta (1996) “Use of viral replicons for the expression of genes in plants,” Mol. Biotechnol. 5:209-221. Selection and construction of vectors and techniques for transforming a wide variety of plant cells are well known, for example, see Hamamoto, U.S. Pat. No. 5,618,699. Alternatively, ABP gene constructs can be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-mediated transformation techniques, including disarming and use of binary vectors, are well described in the scientific literature. Agrobacterium tumefaciens is routinely utilized in gene transfer to dicotyledonous plants. For example, transformants of Arabidopsis thaliana can be generated without using tissue culture techniques by cutting primary and secondary inflorescence shoots at their bases and inoculating the wound sites with Agrobacterium tumefaciens suspensions (Katavic (1994) Mol. Gen. Genet. 245:363-370). To transform Agrobacterium tumefaciens, see, e.g., den Dulk-Ras (1995) Methods Mol. Biol. 55:63-72, and Lin (1995) Methods Mol. Biol. 47:171-178.

For transformation of monocotyledonous plants, including important cereals, see Hiei (1997) Plant Mol. Biol. 35:205-218. See also, e.g., Horsch, Science (1984) 233:496; Fraley (1983) Proc. Natl Acad. Sci USA 80:4803; Thykjaer (1997) supra; Park (1996) Plant Mol. Biol. 32:1135-1148, discussing T-DNA integration into genomic DNA. See also D'Halluin, U.S. Pat. No. 5,712,135, describing a process for the stable integration of a DNA comprising a gene that is functional in a cell of a cereal, or other monocotyledonous plant.

Bombardment-based (ballistic) methodology is another effective means of transforming plant cells. Microprojectile bombardment to deliver DNA into plant cells is an alternative means of transformation for the numerous species considered recalcitrant to Agrobacterium- or protoplast-mediated transformation methods. For example, see, e.g., Christou (1997) Plant Mol. Biol. 35:197-203; Pawlowski (1996) Mol. Biotechnol. 6:17-30; Klein (1987) Nature 327:70-73; Takumi (1997) Genes Genet. Syst. 72:63-69, discussing use of particle bombardment to introduce transgenes into wheat; and Adam (1997) supra, for use of particle bombardment to introduce YACS into plant cells. For example, Rinehart (1997) supra, used particle bombardment to generate transgenic cotton plants. Apparatus for accelerating particles is described U.S. Pat. No. 5,015,580; and, the commercially available BioRad (Biolistics) PDS-2000 particle acceleration instrument; see also, John, U.S. Pat. No. 5,608,148.

The invention also provides for transgenic plants to be used for producing large amounts of the polypeptides of the invention. For example, see Palmgren (1997) Trends Genet. 13:348; Chong (1997) Transgenic Res. 6:289-296 (producing human milk protein beta-casein in transgenic potato plants using an auxin-inducible, bi-directional mannopine synthase (mas 1′, 2′) promoter with Agrobacterium tumefaciens-mediated leaf disc transformation methods).

The nucleic acids and polypeptides of the invention are expressed in or inserted in essentially any plant. Thus, the invention has use over a broad range of plants, including, but not limited to, species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and Zea. In alternative embodiments, the nucleic acids of the invention are expressed in plants which contain fiber cells, including, e.g., cotton, silk cotton tree (Kapok, Ceiba pentandra), desert willow, creosote bush, winterfat, balsa, ramie, kenaf, hemp, roselle, jute, sisal abaca and flax. In preferred embodiments, the transgenic plants of the invention can be members of the genus Gossypium, including members of any Gossypium species, such as G. arboreum;. G. herbaceum, G. barbadense, and G. hirsutum.

One of skill will recognize that after the expression cassette is stably incorporated in transgenic plants and confirmed to be operable, it can be introduced into other plants by sexual crossing. Any of a number of standard breeding techniques can be used, depending upon the species to be crossed. Since transgenic expression of the nucleic acids of the invention leads to phenotypic changes in seeds and fruit, plants comprising the expression cassettes discussed above can be sexually crossed with a second plant to obtain a final product. The seed of the invention can be derived from a cross between two transgenic plants of the invention, or a cross between a plant of the invention and another plant. The desired effects (e.g., expression of the polypeptides of the invention to produce a stronger and/or longer cotton fiber cell) can be enhanced when both parental plants express the ABP polypeptides of the invention.

Transformed Cotton Cells and Transgenic Cotton Plants

In a preferred embodiment, the invention provides transformed cotton plant cells and plants, e.g., from Gossypium, such as G. arboreum;. G. herbaceum, G. barbadense, and G. hirsutum. As discussed above, these can be generated using any of the above transformation techniques. Methods for producing trangenic cotton plants are well known in the art, see, e.g., John, U.S. Pat. Nos. 5,602,321, 5,608,148, 5,597,718, 5,521,078, and 5,495,070; Rinehart (1997) supra.

For example, transduced cotton cells can be produced and cultured to regenerate a whole plant which possesses the transformed genotype and thus the desired phenotype. Such regeneration techniques typically rely on manipulation of certain phytohormones in a tissue culture growth medium, and frequently use a biocide and/or herbicide marker which has been introduced together with the desired nucleotide sequences. See, e.g., Rangan, U.S. Pat. Nos. 5,834,292, and 5,695,999, describing methods for the regeneration of cotton plants from somatic cells. The methods include providing a cotton explant; culturing the explant in a callus growth medium supplemented with glucose as a primary carbon source until the secretion of phenolic compounds has ceased and undifferentiated callus is formed from the explant; culturing the undifferentiated callus in callus growth medium supplemented with sucrose as a primary carbon source until embryogenic callus is formed from the callus; transferring the embryogenic callus to a plant germination medium, culturing the embryogenic callus on the plant germination medium until a plantlet is formed from the embryogenic callus, transferring the plantlets to soil, growing the plantlets to produce seeds from self pollination, collecting the seeds, planting the seeds, growing the seeds under conditions to select for a desired characteristic and collecting the plants with the desired characteristics.

Alternatively, a mature plant can be innoculated with ABP-expressing nucleic acid using, e.g., bombardment techniques, as described above (see, e.g., Rinehart (1996) supra). See also Bowen, U.S. Pat. No. 5,736,369, describing methods of generating transgenic cereal plants which are stably transformed by biolistic bombardment in order to target non-differentiated meristem cells for transformation.

Stable integration and expression of foreign genes in cotton plants has been demonstrated and repeated using Agrobacterium-mediated transformation of cotton plant cells, see, e.g., Umbeck (1987) Bio/Technology 5:263-266; Firoozabady (1987) Plant Mol. Biol. 10:105-116. The transformation of cotton tissues is accomplished by Agrobacterium infection and regeneration. See also Finer (1990) Plant Cell Rep. 8:586-589; McCabe (1993) Bio/Technology 11:596-598.

Plant regeneration from cultured protoplasts, including cotton protoplasts, is described, e.g., in Evans, PROTOPLASTS ISOLATION AND CULTURE, HANDBOOK OF PLANT CELL CULTURE, pp. 124-176, Macmillian Publishing Company, New York, 1983; Binding, REGENERATION OF PLANTS, PLANT PROTOPLASTS, pp. 21-73, CRC Press, Boca Raton, 1985. Regeneration can also be obtained from plant callus, explants, organs, or parts thereof. Such regeneration techniques are described generally in Klee (1987) Ann. Rev. of Plant Phys. 38:467. See also, e.g., Cheng (1995) Methods Mol. Biol. 55:181-188; Hampp (1997) Planta 203 Suppl: S42-S53, for leaf protoplasts; Ruesink (1979) “Fusion of higher plant protoplasts,” Methods Enzymol. 58:359-367.

Examination of Cotton Fibers In Transgenic Plants

After generating transduced cells and transgenic cotton plants expressing one or more ABP polypeptides of the invention, the cells and plants are analyzed for expression of recombinant ABP and altered cotton fiber characteristics. There are various methods of measuring fiber characteristics. For example, fiber strength is a factor in determining yarn strength. Fiber with superior strength is preferred in manufacturing processes. Cotton fiber strength can be measured in a number of ways. The most common measurement is that of the fiber bundle strength.

In one exemplary technique, fiber bundle strength measurements are made with a ⅛ inch spacer between the clamp jaws (⅛ inch gauge) of a Stelometer or a Motion Control High Volume Instrument (HVI). The results are given in grams per tex. A tex unit is equal to the weight in grams of 1,000 meters of the material. Results of Stelometer ⅛ inch gauge tests are calculated using standard formulas. The results are adjusted to Pressley level by the use of calibration cottons.

Alternatively, fiber length can be used to demonstrate the fiber-strengthening effects of the compositions and methods of the invention. Comb sorters provide a way of sorting the fibers into different length groups, usually 1/16 of inch intervals. Instruments such as a fibrograph and HVI system can be used to compute length in terms of “Mean” and “Upper Half Mean” length. The mean is the average length of all fibers and the upper half mean (UHM) is the average length of longer half of the fiber distribution. The fibrograph measures length in span lengths at a given percentage point. For example, the 2.5% span length is the span length that agrees best with classers staple and indicates that 2.5% of the fibers are of this length or longer.

Another quantitatively measurable criteria is fiber fineness and maturity. They can determined by the “micronaire test.” This is an instrument test which measures the resistance of a plug of cotton to air flow. In one exemplary protocol, from 47 to 52 grains of cotton are placed in the instrument specimen holder and compressed to a fixed volume. Air at a known pressure is forced through the specimen. The amount of flow is indicated by a direct reading scale. The readings obtained are a relative measurement of either the weight per unit length or cross-sectional size of the fibers. Because the instrument measurements may differ from the actual weight per inch, depending up on the fiber characteristics of the sample, the results are reported in terms of “micronaire reading” instead of micrograms per inch. The air flow reacts to the surface area of the fibers presented to it. Because both small diameter mature fiber and a large diameter thin walled fiber will present a relatively high surface area, the test will indicate both maturity and fineness. The fiber diameter within a given variety is fairly consistent. Therefore the micronaire index will more likely indicate maturity variation than variations in fineness (fiber maturity is defined as the total cell wall thickness related to the diameter or width of the fiber; a mature fiber is one in which twice the cellulose wall thickness equals or exceeds the width of the lumen).

An arealometer is another means to quantitatively measure changes in cotton fiber quality and quantity. An arealometer is an air flow instrument responsive to specific area and immaturity ratio. Thus, it is used to measure fiber fineness and immaturity. Specific area (A) is defined as the ratio of the external surface of the fibers to the volume of fibrous material; and immaturity ratio (I) is defined as the area of a circle having the same perimeter as an average fiber to the actual cross section area of the fiber (see, e.g., Hertel and Craven (1951) Textile Research J. 21:765-774). Other useful parameters calculated or measured by arealometer include perimeter (p), weight fineness in terms of area density of cellulose (W), and wall thickness (t). The increase in apparent specific area produced by compression in Arealometer (D) is related to I.sup.2.

Production Of Anti-Abp Antibodies

The ABP peptides and polypeptides of the invention can also be used to generate an immune response. Anti-ABP antibodies and antiserum are useful for quantitative and qualitative measurement of ABP polypeptide expression in vivo and in vitro in natural and recombinant systems, including transgenic plant expression in vivo.

Such antibodies can be generated in vitro, e.g., using recombinant antibody binding site expressing phage display libraries, or in vivo, e.g., using animals. The peptide can be conjugated to another molecule or can be administered with an adjuvant. Alternatively, DNA (e.g., expression cassette, vector) encoding a polypeptide comprising any ABP epitope can be directly administered to the animal selected to generate the anti-ABP antibody or antiserum (the ABP coding sequence is part of an expression cassette or vector capable of expressing the immunogen in vivo, see, e.g. Katsumi (1994) Hum. Gene Ther. 5:1335-9).

Methods of producing polyclonal and monoclonal antibodies are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Anti-ABP antibodies of the invention can also be generating using transformed or trangenic plants; see, e.g., Verch (1998) J Immunol Methods 220:69-75, who used a tobacco mosaic virus-based vector to express monoclonal antibody directed to a human colon cancer antigen in tobacco plants. Genes encoding heavy and light chains of this antibody were introduced independently into the tobacco mosaic virus vector. See also, Ma (1994) Eur J Immunol 24:131-8; Hiatt (1992) FEBS Lett 307:71-5.

As noted above, ABP reactive antibodies can also be generated from libraries of recombinant antibodies displayed on phage (“phage display libraries”) or on cells. See, e.g., Huse (1989) Science 246:1275; Ward (1989) Nature 341:544; Hoogenboom (1997) Trends Biotechnol. 15:62-70; Katz (1997) Annu. Rev. Biophys. Biomol. Struct. 26:27-45. Recombinant antibodies can also be expressed by transient or stable expression vectors in mammalian cells, as in Norderhaug (1997) J. Immunol. Methods 204:77-87; Boder (1997) Nat. Biotechnol. 15:553-557.

The ABP reactive antibodies of the invention are used as reagents and methods in variety of antibody based assays. Immunological binding are well known in the art; see, e.g., U.S. Pat. Nos. 4,366,241; 4,376,110; 4,517,288; and 4,837,168; METHODS IN CELL BIOLOGY Vol. 37., Antibodies in Cell Biology, Asai, ed. Academic Press, Inc. New York (1993); Sambrook, Stites; Silzel (1998) Clin. Chem. 44:2036-43; Rongen (1997) J. Immunol. Methods 204:105-133.; Hashida (1995) Biotechnol. Annu. Rev. 1:403-51; Bao (1997) J. Chromatogr. B. Biomed. Sci. Appl. 699:463-80; Self(1996) Curr. Opin. Biotechnol. 7:60-5. See also, Lough (1998), “Western analysis of transgenic plants,” Methods Mol Biol 1998;81:447-51; Fido (1995) Methods Mol Biol 49:423-37.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

The following examples are offered to illustrate, but not to limit the claimed invention.

### Example 1

### Cloning of Auxin Binding Polypeptide (ABP)-Encoding Sequences

The invention provides for a genus of auxin binding polypeptide (ABP-encoding) nucleic acids. The following example details the cloning and characterization of these sequences.

Plant Materials

Upland cotton (Gossypium hirsutum L. cv. Acala SJ-2) was grown in the greenhouse under a 27° C./21° day/night temperature regime. Developing cotton bolls were collected at specific stages after anthesis from flowers tagged an anthesis (0 day post-anthesis: dpa). Determination of the developmental stages of ovules before anthesis was abased on the phylloctactic arangement of cotton flowering nodes relative to the position of opended flowers at anthesis. Ovules were collected from developing flowers at −9, −6, −3, −1, 0, 1, 3, 5, 10, 15, 20, 25, 30, and 35 dpa. The collected ovules were frozen in liquid nitrogen and stroed at −80° C. for RNA isolation.

Preparation of Probe for Library Screen

PCR was used to prepare a hybridization probe to use for screening an unamplified cDNA library to isolate a full-length cotton ABP clone. The selection of target sequences for amplification was based on a homology comparison of deduced ABP amino acid sequences from maize, Arabidopsis, tobacco and strawberry to identifed conserved regions. The comparison analysis was performed using the “CLUSTAL” analysis using the PC/GENE software program (IntelligGenetics, Inc., Mountain View, Calif.). Two degenerate primers, COT126 (5′-CACAGGCAYTCHTGT-3′) and COT127 (5′-GCRGCHGTRTGWGGCAT-3′), which were targeted to the highly conserved regions HRHSCEEVF and MPHTAA, respectively of the ABP sequence were synthesized. The symbol Y in the COT primer sequence indicates a mixture of C/T nucleotides; H indicates a mixture of A/C/T, R indicates a mixture of A/G, and W indicates a mixture of A/T.

PCR amplification was performed using 25 μl of recombinant phage (3.4×10⁷ pfu/μl) from an unamplified −3 dpa λgt10 cotton ovule cDNA library (Wilkins, Plant Physiol. 102:679-680, 1993) as the DNA template. The reaction contained 200 μM dNTPs, 25 pmol of each COT primer, and 2.5 units of Taq DNA polymerase (Promega, mMadison, Wis.) in a buffer supplied with the enzyme in a reaction volume of 125 μl, which contained a final MgCl₂ concentration of 1.5 mM that was supplied by the phage storage buffer. Reactions were conducted in an Ericomp (San Diego, Calif.) temperature cycler for 30 sec at 94° C., followed by 30 cycles of 92° C. for 2 min, 42° C. for 2 min, and 72° C. for 2 min with a final extension step for 10 min at 72° C. A 260 bp PCR amplification product was isolated from the gel and cloned directly into the vector PCR2.0 (Invirogen, Carlsbad, Calif.). The identity of the fragment as corresponding to the conserved region of ABP was confirmed by DNA sequencing.

Isolation and Characterization of Developmental-Specific ABP cDNA Clones

A full-length cDNA was obtained by screening an amplified 0 dpa ovule λgt10 cDNA library at high stringency using the probe to the conserved ABP-1 region that was generated by PCR. This cDNA, ABP0, was then used to probe a Uni-Zap XR 10 dpa cotton fiber cDNA library (Stratagene, La Jolla, Calif.) to identify fiber ABP cDNA(s).

### Example 2

### Inducing Auxin Binding Protein-Expressing Transgenic Plants to Express Cotton Fibers of Increased Length and Strength

A further embodiment of the invention provides for expression of auxin binding polypeptide (ABP)-encoding nucleic acid of the invention in transgenic plants using, e.g., constitutive or inducible, tissue-specific, developmentally specific, or environmentally sensitive transcriptional control elements, such as promoters and enhancers. The following example details the induction of trangenic cotton plants to produce cotton fibers of increased length.

The coding sequence for the auxin binding polypeptide (SEQ ID NO:1) can be expressed under the control of a constitutive or inducible promoter. An exemplary constitutive promoter is the 35S promoter (see, e.g., Mengiste (1997) supra). An exemplary inducible promoter is a viral sub-genomic promoter, e.g., from the tomato bushy stunt virus (see, e.g., Hillman (1989) supra; GenBank Accession Nos. M21958, M31019, U80935). An exemplary tissue (cotton fiber)- and developmentally-specific promoter is the FbL2A (cotton plant) promoter, as described by Rinehart (1996) Plant Physiol. 112:1131-1141. The gene expression cassette, including the ABP coding sequence, selected inducible or constitutive promoter, and optionally, tagging, selection and marker genes (described in detail, above), are cloned into an appropriate plasmid, as described, e.g., by Rinehart (1996) supra, using conventional techniques. The ABP expressing plasmids were introduced into cotton seed axes; the resultant transformed plants were analyzed for stable integration and expression of the transgene; and the plants were assessed for germline versus epidermal transformation, also as described by Rinehart (1996) supra. The progeny of the epidermal transformants will not inherit the transgene; they are vegetatively propagated.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

```
SEQ ID NO: 1
        GACCTTGCTT CATTTTCTTC TTCCTTCTCT TAAACTTGCT TCCATTCTTT CGAACTCTCG
        AAGCTTCTCA CTGCTCCATC AAAGGGTTAC CTCTGGTGAG GAACATTGCT GATCTTCCAC
        AGGATAATTA TGGAAGAGGA GGTTTATCCC ATATAACTGT TGCTGGTTCT CTCTTGCATG
        GGTTGAAAGA AGTTGAGGTT TGGCTTCAAA CATTTGCACC AGGATCGCGC ACGCCGATCC
        ATAGGCACTC TTGTGAAGAA GTTTTTGTTG TTCTCAAGGG CAGTGGCACT CTATATCTCG
        CCTCGAGTTC TAATAAGTAC CCTGGAAAAC CGGAGGAGCA CTTTATATTT TCGAATAGCA
        CGCTTCATAT CCCTGTCAAT GATGTTCACC AGGTCTGGAA TACAAATGAA CATGAAGATT
        TGCAAATGCT TGTGATAATA TCTCGGCCGC CTATCAAAGT GTTCATATAT GAAGATTGGT
        TGATGCCTCA CACTGCAGCT AAGTTGAAGT TTCCCTACTA TTGGGATGAG CAGTGCTTTC
        AAGTACCTCA GAAAGATGAG CTTTAATTTT TGAAGACACG CCCCTTCACA TGCTACTATA
        TGAGCACTGT AATGGGGCCA TTCCCATTTT ACTGCTCAGA TTACTTTACA AATTACATAA
        AGATTACAAC ATCTTAGCTT AGTTTGTATA TTTTCCCCCT CATTTGAAGT CTGAATCCAT
        TTTCTATTTT CATTTCAAAA AAAAAAAA
```

ID ABP0P PRELIMINARY; PRT; 156 AA.

DT SEP. 7, 1999 (CREATED BY PC/GENE PROGRAM TRANSL)

DE MELD CREATED BY ASSEMGEL REV 2.0

CC TRANSLATED FROM DNA SEQUENCE ABP0 (BASES 96 TO 563).

SQ SEQUENCE 156 AA; 17881 MW; 138224 CN;

VRNIADLPQD NYGRGGLSHI TVAGSLLHGL KEVEVWLQTF APGSRTPIHR HSCEEVFVVL KGSGTLYLAS SSNKYPGKPE EHFIFSNSTL HIPVNDVHQV WNTNEHEDLQ MLVIISRPPI KVFIYEDWLM PHTMKLKFP YYWDEQCFQV PQKDEL

What is claimed is:

1. An isolated nucleic acid comprising a nucleic acid sequence encoding a polypeptide capable of binding to auxin, wherein the nucleic acid sequence hybridizes to SEQ ID NO:1 under stringent hybridization conditions or encodes a polypeptide having a sequence as shown in SEQ ID NO:2.

2. The isolated nucleic acid of claim 1, wherein the stringent hybridization conditions comprise a salt concentration of about 0.02 molar at pH 7 and a temperature of about 60° C.

3. The isolated nucleic acid of claim 1, where the nucleic acid has a sequence as set forth in SEQ ID NO:1.

4. An expression cassette comprising a nucleic acid sequence operably linked to a promoter, wherein the nucleic acid sequence hybridizes to SEQ ID NO:1 under stringent hybridization conditions, or encodes a polypeptide having a sequence as shown in SEQ ID NO:2.

5. A vector comprising a nucleic acid sequence operably linked to a promoter, wherein the nucleic acid sequence hybridizes to SEQ ID NO:1 or encodes a polypeptide having a sequence as shown in SEQ ID NO:2.

6. A transgenic plant cell comprising a heterologous nucleic acid sequence operably linked to a promoter, wherein the heterologous nucleic acid sequence hybridizes to SEQ ID NO:1 under stringent hybridization conditions or encodes a polypeptide having a sequence as shown in SEQ ID NO:2.

7. A transfected cell comprising a nucleic acid encoding an auxin-binding polypeptide and a non-naturally occurring nucleic acid sequence,
wherein the heterologous nucleic acid sequence hybridizes to SEQ ID NO:1 under stringent hybridization conditions, and,
the auxin-binding polypeptide, upon expression in a plant cell, is capable of binding to plant auxin.

8. A transgenic plant comprising a heterologous nucleic acid sequence operably linked to a promoter, wherein the heterologous nucleic acid sequence hybridizes to SEQ ID NO:1 under stringent hybridization conditions or encodes a polypeptide having a sequence as shown in SEQ ID NO:2.

9. The transgenic plant of claim 8, wherein the promoter is a constitutive promoter and the nucleic acid is constitutively expressed.

10. The transgenic plant of claim 8, wherein the promoter is a tissue specific promoter.

11. The transgenic plant of claim 10, wherein the tissue specific promoter is primarily active in cotton fiber cells.

12. The transgenic plant of claim 8, wherein the promoter is developmentally regulated.

13. The transgenic plant of claim 12, wherein the developmentally regulated promoter is primarily active in late primary and early secondary wall synthesis stages.

14. A transgenic plant, or progeny thereof, into which a heterologous nucleic acid sequence which hybridizes to SEQ ID NO:1 under stringent hybridization conditions has been introduced, wherein the nucleic acid encodes an auxin-binding polypeptide,
wherein the auxin-binding polypeptide, upon expression in a plant cell, is capable of binding to plant auxin.

15. The transgenic plant of claim 8 or 14, where the nucleic acid has a sequence as set forth in SEQ ID NO:1.

16. The transgenic plant of claim 8 or 14, wherein the stringent hybridization conditions comprise a salt concentration of about 0.02 molar at pH 7 and a temperature of about 60° C.

17. The transgenic plant of claim 8 or 14, wherein the plant is a member of the genus Gossypium.

18. The transgenic plant of claim 17, wherein the Gossypium plant is a Gossypium specie selected from the group consisting of G. arboreum;. G. herbaceum, G. barbadense, and G. hirsutum.

19. A method for detecting an auxin binding protein-encoding nucleic acid in a nucleic acid-containing biological sample, the method comprising the following steps:
(a) contacting the sample with a nucleic acid of claim 1,
(b) hybridizing the nucleic acid of claim 1 to the nucleic acid in the sample; and,
(c) detecting hybridization of the nucleic acids.

20. The method of claim 19, wherein the biological sample can comprise a plant cell.

21. The method of claim 20, wherein the plant cell is a cotton plant cell selected from the group consisting of G. arboreum;. G. herbaceum, G. barbadense, and G. hirsutum.

22. The method of claim 19, wherein the nucleic acid of claim 1 comprises an oligonucleotide primer pair capable of amplifying a subsequence of the nucleotide of claim 1.

23. The method of claim 19, wherein detecting the hybridization of the nucleic acids comprises detection of an amplification product.
